(19) United States
(12) Patent Application Publication
(10) Pub. No.: US 2008/0081364 A1
(43) Pub. Date: Apr. 3, 2008

(54) Preservation system for stratabound microorganisms

(76) Inventors: Richard M. Greenfield, Ft. Pierce, FL (US); Betsey G. Moore, Ft. Pierce, FL (US)

Correspondence Address: MCHALE & SLAVIN, P.A., 2855 PGA BLVD, PALM BEACH GARDENS, FL, 33410, US

(21) Appl. No.: 11/538,270

(22) Filed: Oct. 3, 2006

(51) Int. Cl. C12M 1/00

(52) U.S. Cl. 435/307.1

(57) ABSTRACT

The present invention is directed towards a storage system for biologically viable aquatic microorganisms. The system includes substrate with hydrated and viable microorganisms attached thereto, and a packing material which envelops a volume of the substrate in a manner effective to preserve a degree of hydration necessary to maintain said microorganisms viable during a storage period therein. At least a portion of the package material is constructed and arranged to permit gaseous exchange with the exterior atmosphere to maintain aerobic conditions throughout packaged volume of substrate to prevent spoilage and putrefaction therein. These viable bio-active microorganisms are then delivered to a closed aquatic environment to begin biochemical cycling therein.

### FIELD OF THE INVENTION

This invention relates to promoting biochemical cycling in an aquatic habitat; particularly, to a storage system capable of delivering beneficial and viable microorganisms to a closed aquatic environment; and most particularly, to a storage system useful for effectively maintaining beneficial microorganisms in a viable and bio-active state for extended periods of time prior to the introduction into an aquarium.

### BACKGROUND OF THE INVENTION

Aquariums have been popular for many years for both novice and expert aquarists alike. The typical aquarium contains a diverse mix of marine life, including aquatic plants, fish, and invertebrates. As the popularity of both marine and freshwater aquariums continues to grow, the number of retail products designed to assist the aquarist establish and maintain a healthy aquatic habitat also increases.

Unlike the essentially open system of oceans and lakes, fish excrement and decaying food inside the closed system of an aquarium can quickly accumulate, especially in new aquariums. This accumulation of organic material increases the levels of toxic nitrogenous compounds, such as ammonia and nitrite, beyond that which can be tolerated by the inhabitants, creating a stressful or even fatal environment. In order to maintain a healthy environment the level of these harmful compounds must remain low. This often requires frequent water changes and the use of complex chemical and/or mechanical filtration systems. Chemical filtration systems include, for example, activated carbon to remove dissolved organic compounds. Mechanical filtration systems assist in the collection of the solid wastes and excess food. However, neither mechanical nor chemical filtration techniques are particularly effective in removing toxic nitrogenous compounds (ammonia and nitrite).

While ammonia and nitrite are toxic to fish at very low levels (several part-per-million (ppm)), the fish are able to tolerate several hundred ppm of nitrate. Fortunately, beneficial microorganisms (nitrifiers) are able to convert harmful ammonia into tolerable nitrates through a process called nitrification. Examples of these beneficial microorganisms which drive this nitrification process include aerobic bacteria Nitrosomonas and Nitrobacter. First, Nitrosomonas oxidize the ammonia to nitrite ions and water. The nitrite-rich metabolic waste of these microorganisms is then available as a food source for Nitrobacter, which oxidize the nitrite ions to nitrate. Aquatic plants and algae utilize nitrate as a fertilizer and during plant respiration, thereby keeping nitrate levels at an acceptable level in the water.

The aforementioned nitrification process is but a portion of a larger biochemical cycle. Biochemical cycling is the process of establishing an equilibrium between the synthesis of ammonia as a result of waste production and decay of organic constituents, and its ultimate conversion into nitrate by the beneficial microorganisms in situ. However, establishing conditions for biochemical equilibrium within a pristine aquarium may take upwards of six weeks depending on the aquarium type (freshwater or marine), and conditions (pH, temperature, bioload, light conditions, etc). This slow initial cycling period is due to the slow rate at which the microorganisms reproduce and establish a biofilm (the matrix created by the microorganisms on a substrate in which they reside). It is during this initial cycling period that the aquarium inhabitants are most vulnerable.

When fish or other animals are added to an aquarium, a small number of these beneficial microorganisms are often attached and begin colonizing the aquarium. However, the number of animals introduced often overwhelms the nascent colonies of microorganisms and the ammonia level quickly rises to a toxic level. Accordingly, commercial products have been developed that attempt to quickly establish biochemical cycling (equilibrium) in the closed system by introducing cultures of these beneficial nitrifying microorganisms into the aquarium.

One such product includes dried or lyophilized microorganism cultures that are introduced into the aquarium. However, the process of drying the microorganisms kills most if not all of the colonies. The addition of these putrefied microorganisms into the aquarium by the consumer only supplies more decaying material therein which inadvertently increases ammonia production. Any microorganisms that do remain viable after the drying process are induced into a dormant state by the process, that is, they are not metabolically and physiologically active (i.e., bio-active). This dormancy prevents the consumption and depletion of cellular food reserves and air during storage inside the sealed container. However, when added to the aquarium the dormant organisms will require several days to become active again. As discussed above, during this initial cycling period the ammonia levels can accumulate to toxic levels.

Similarly, liquid cultures containing beneficial microorganisms have been packaged and stored in fluid-impermeable (air-tight) containers until opened for use. However, if these liquid cultures often contain other species of bacteria, particularly anaerobic varieties, the beneficial bacteria will die from the toxic effects of the waste (hydrogen sulfur) produced by the anaerobic bacteria. Under these conditions the anaerobic bacteria will flourish and cause the package contents to discolor and produce a sulfur “rotten-egg” smell making the product undesirable for commercial use. Moreover, the addition of large amounts of these anaerobic bacteria in the aquarium can stress or kill the inhabitants. Even if no other types of bacteria are present in the sealed container, the aerobic beneficial microorganisms often expire due to the limited supply of oxygen inside the container and/or exposure to their own waste products.

Packages containing the aforementioned microorganisms have been refrigerated for long term storage. However, the low temperatures (usually less than 4 degrees C.) induce a state of dormancy in the microorganisms, which is undesirable for the reasons set forth above. Obviously, it is difficult to store packages containing microorganisms under continuous refrigeration, since these packages must be loaded into refrigerated delivery trucks and warehouses. Once refrigerated, the microorganisms must remain cool since the shelf-life of these microorganisms decreases rapidly once they are returned to room temperature. Therefore, this refrigeration process makes the end product expensive and impractical.

The instant invention satisfies a long felt need for a practical and economical storage system that will maximize the viability of bioactive microorganisms, illustrated by, albeit not limited to aquatic microorganims required to begin immediate biochemical cycling upon introduction into a closed aquatic system, while minimizing the packaging and shipping cost normally associated with such products.

### PRIOR ART

Although there are numerous patents and publications directed to air-tight packaging for storing beneficial nitrifying microorganism, none of the known prior art teach a storage system which provides for gaseous exchange between the interior of the container and the ambient atmosphere while eliminating the need for excess water addition so as to reduce spoilage and putrefaction therein for extended periods of time at room temperature.

U.S. Pat. Nos. 6,939,708 and 6,376,229, both to Morris et al., disclose a method of rapid biochemical cycling of aquariums using naturally preserved granular marine substrate material. The method describes the steps of harvesting marine sand from a natural submerged marine environment. The sand contains marine microorganism in the form of a natural organic biofilm. The sand and the microorganisms are packaged together with an optimal volume of water and headspace air inside the retail container. The container is specifically dimensioned and configured for maintaining the microorganism in a bio-active state for prolonged periods at room temperature.

Unlike the present invention, the retail package disclosed in patents '708 and '229 requires specific parameters in order to sustain the marine organisms in a bio-active state. Specifically, the harvested material must form a uniform layer between ½-inch and 3-inches in depth inside the package. This 3-inch depth maximum is deemed critical since it allows the batch volume of water and headspace gas sealed inside the container to diffuse through the harvested material to preserve the respiratory cycle of the microorganisms. However, should the harvested material inside the container exceed this depth, only microorganisms within the 3-inch region remain biologically viable. Therefore, the consumer may be inadvertently adding putrefied microorganisms into the aquarium, with a concomitant increase in the level of nitrogenous compounds therein. Moreover, since the microorganisms are sealed in an airtight container, the invention of the '708 patent is limited to a specific range of ratios of volumes of air, water, and sand necessary to maintain the viability of the microorganisms.

This is in direct contrast to the present invention, which maintains aerobic conditions throughout the entire volume of packaged substrate while preventing dehydration of the microorganisms therein. This acts to prevent putrefaction of the beneficial microorganisms stored therein. Since the package of the instant invention is not limited to the aforementioned parameters found in the '708 and '229 patents, the product may be manufactured in various ways (less storage space needed). Furthermore, since the package of the present invention is able to vent continuously, the package is less likely to rupture or burst during shipping.

U.S. Pat. No. 6,101,685 to Archibald et al., discloses a container for storing fine particles in a sealed package, wherein air entrapped during the filling of the container can be expelled through microscopic pores by compression without loss of the fine particles. In contradistinction to the present invention, the container of Archibald et al., is designed to minimize moisture accumulation and the introduction of air therein in an attempt to prevent microbe proliferation inside the container.

U.S. Pat. No. 5,733,774 to Jin et al., is directed toward stabilized bacterial formulations which can survive long term storage at high temperatures. The bacteria are dried by a suitable method (air-drying, vacuum drying, etc.) until they reach a dormant state. The oxygen is then removed from the environment surrounding the bacteria and the bacteria is packaged and stored in material impermeable to gas and water vapor until such time as it is ready for use.

U.S. Pat. No. 5,492,705 to Porchia et al., discloses a flexible film and flexible food storage bag for packaging produce such as vegetable and fruits, wherein the film or bag has a plurality of micro-holes specifically designed to allow the produce therein to respire at a controlled rate such that localized condensation and weight loss is minimized. Unlike the present invention, the storage bag of Porchia is designed to reduce microbial growth therein and prevent produce mushiness (softness).

U.S. Pat. No. 4,874,707 to Bock, discloses a complex laboratory process for creating an aqueous suspension of nitrifying bacteria using a growth medium containing ammonia or nitrite. The process introduces air, pure oxygen, or a mixture of air and pure oxygen through a gas permeable non-porous tube which is submerged in the growth medium. Unlike the present invention where aerobic conditions are maintained in the interstitial spaces of the substrate to prevent putrefaction of the beneficial microorganisms and proliferation of noisome anaerobic microbes, the nitrifying bacteria of the '707 patent remain submersed in the continuous phase of the growth media, thus, aerobic conditions exist only near the tube surface.

U.S. Pat. No. 5,314,542 to Cassidy et al., discloses a culture of Nitrosomonas cells that are isolated from the ocean or fresh water depending on the desired end use. The cultures are collected and concentrated using centrifugation, filtration or other means. The concentrate is then re-suspended in sterile water and packaged in sterile, air-tight containers. The cells enter into a metabolic state of inactivity (dormancy), but will remain viable (living) for at least one year. The preserved cells are returned to their metabolically active state by adding ammonia to the opaque container and leaving the container open (without the cap) in a dark place for 72 hours prior to addition into the aquarium.

Thus, the present invention satisfies a longfelt need by providing what has heretofore been lacking in the art, a packaging system that will deliver the maximum amount of viable beneficial microorganisms with the least amount of spoilage and putrefaction. Furthermore, because the present invention does not require specific parameters or nutrients to maintain microorganism respiration throughout the packaged substrate, numerous packaging options are made available to the manufacturer for display and retail sale.

### SUMMARY OF THE INVENTION

The present invention provides a unique storage system for a substrate containing biologically viable microorganisms, illustrated by, albeit not limited to aquatic microorganisms. The system is comprised of a substrate including viable microorganisms and a breathable packaging material which envelops a volume of the substrate in a manner effective to preserve the requisite degree of hydration necessary to maintain the microorganisms viable during storage. At least a portion of the package material is constructed and arranged to permit gaseous exchange with the exterior atmosphere to maintain aerobic conditions essentially throughout the volume of substrate, thereby preventing spoilage and putrefaction therein. Upon removal from the packaging material, the bio-active microorganisms are delivered to a closed environment, e.g. an aquatic environment, where they readily assist in biochemical cycling therein.

The instant invention embodies a storage system for containing a substrate bound population of microorganisms, and maintaining biological viability and hydration thereof, and comprises in combination a substrate constructed and arranged to bind a plurality of hydrated and viable microorganisms thereon; and a breathable packaging material which forms a containment device effective to envelope and contain a volume of said microorganism bound substrate, wherein said containment device defines an interior space, and is constructed and arranged to enable gaseous exchange therethrough of ambient atmospheric gases, e.g. oxygen in the ambient atmosphere and carbon dioxide within the containment device, in a manner effective to maintain a gaseous environment and a degree of hydration necessary to ensure said microorganisms remain viable during storage. In a preferred, albeit non-limiting embodiment, at least a portion of said packaging material is constructed and arranged so that it provides gaseous exchange between said ambient atmospheric gases and said interior space, so as to maintain aerobic conditions throughout said volume of substrate, and thereby prevent death and putrefaction of said bound microorganisms.

Illustrative, albeit non-limiting examples of microorganism contemplated for use in the present invenion include those selected from marine genera, such as Pseudomonas, Vibrio, Bacillus, Nitrosomonas, Nitrobacter, Desulfococcus, and Methanolobus. Microorganisms classified as those useful for aerobic starch hydrolysis, anaerobic starch hydrolysis, sulfate reduction, gelatin liquification, cellulose decomposition, aerobic nitrate reduction, anaerobic nitrate reduction and nitrification, are further contemplated as being useful in the instant invention.

Further contemplated as useful in the instant invention are nitrogen fixing bacteria such as Azotobacter, Azospirillum, Agrobacterium, and Gluconobacter, humate consuming bacteria such as Actinobacteria, and sulfur oxidizers. Additionally, leaf litter decomposers such as fungi selected from the genera Alatospora, Anguillospora, Clavatospora, Flagellospora, Heliscus, Lemonniera and Tricladium, and crop disease suppressors such as Bacillus megaterium and Pseudomonas fluorescens are contemplated.

According to one embodiment, at least a portion of the breathable packaging material includes a plurality of gas permeable openings or perforations which are sized to permit permeation of gas yet prevent permeation of liquid, such as by capillary action, wicking, or the like. The breathability of the package effectively maintains the microorganisms in a viable and bio-active state by permitting continuous gaseous exchange between the ambient atmosphere and the interior of the package, while maintaining a sufficient degree of hydration therein.

Accordingly, it is an objective of the instant invention to teach a storage system that will effectively and efficiently deliver to the aquarist, or the like end user, the maximum amount of viable and bioactive microorganisms needed to begin biochemical cycling when added to a particular environmental system, such as a closed aquatic system.

It is a further objective of the instant invention to teach a storage system wherein the microorganisms are preserved for an extended period of time (in excess of 12 months) at approximately room temperature without the need for additional enrichment media, nutrients or moisture.

It is yet another objective of the instant invention to provide a storage system which does not require the addition of specific ratios of substrate, water and air inside the package prior to sealing, to accommodate microorganism respiration; this reduces the “dead” weight of water inside the sealed package and reduces shipping cost.

Another objective of the instant invention is to teach a storage system which does not promote the growth of anaerobic bacteria therein.

Still another objective of the present invention is to teach a storage system that is resistant to rupture or bursting.

Other objects and advantages of this invention will become apparent from the following description, wherein are set forth, by way of illustration and example, certain embodiments of this invention.

### DEFINITIONS

The following list defines terms and phrases used throughout the specification. Although the terms and phrases are listed in a singular tense the definitions are intended to encompass all grammatical forms.

As recited herein, the terms “aquatic system” and “aquatic environment” are used interchangeably herein to refer to a saltwater system similar to that of natural seawater, or a freshwater system similar to that found in river or lake environments.

As recited herein, the term “beneficial microorganisms” refer to any aquatic aerobic organisms responsible for turning toxic nitrogenous compounds into less harmful nitrites and nitrates. These microorganisms may be engineered in a laboratory or may be indigenous microorganisms harvested from marine (saltwater, brackish, etc.,) or freshwater environment. “Beneficial microorganisms” further refer to any microorganiasms useful for treating a controlled environment such as a terrarium or the like, as outlined above.

As recited herein, the term “bioactive” refers to the physiological and metabolic activity of the microorganism.

As recited herein, the term “viable” refers to living organisms.

As recited herein, the term “biofilm” refers to an immobilized matrix of cells that over time cover all submerged objects and surfaces inside the closed aqueous system. The matrix is produced and inhabited by microorganisms which carry out certain biochemical processes, e.g., the nitrification process.

As recited herein, the term “substrate” refers to any suitable material to which the microorganism can attach, grow and form a biofilm. Some non-limiting examples of suitable substrates include at least one of aragonite, sand, gravel, coral, rock, pebbles, clay, fibrous material, or the like. The substrate may be naturally occurring and harvested from marine or freshwater systems. Otherwise, the substrate may be manufactured.

As recited herein, the term “breathable packaging material” refers to any sufficiently durable and tear resistant material that is gas permeable, yet precludes permeation of liquid therethrough. One non-limiting example of breathable packaging material includes a package material capable of having a plurality of appropriately sized perforations formed therein using any suitable technique. Another example, of a breathable packaging material includes a polymer (alone, compounded, or blended with other polymers or additives) with inherent physical properties that make it liquid-impermeable and gas-permeable. Some non-limiting examples of suitable polymers include LDPE (low density polyethylene), PVC (polyvinyl chloride), EVA (ethylene vinyl acetate co-polymer), PORDIX (produced by OhE Chemicals, Inc.) and AFFINITY GA POPs (manufactured by Dow Chemical Company). At least a portion of the breathable packaging material may be opaque, semi-opaque, transparent, or translucent. Moreover, the breathable packaging material may be flexible, semi-flexible, or rigid.

### BRIEF DESCRIPTION OF THE FIGURES

FIG. 1 is an upper perspective view of one preferred embodiment of the present invention partially cut away showing the package filled with substrate;

FIG. 2 is a sectional view of the package taken along lines 3-3 of FIG. 1.

FIG. 3 is an enlarged view of one embodiment of a packaging material having a perforation formed therein; and

FIG. 4 is an enlarged view of the substrate inside the package showing the layers of biofilm having a thin film of liquid thereon.

### DETAILED DESCRIPTION OF THE INVENTION

Detailed embodiments of the instant invention are disclosed herein, however, it is to be understood that the disclosed embodiments are merely exemplary of the invention, which may be embodied in various forms. Therefore, specific functional and structural details disclosed herein are not to be interpreted as limiting, but merely as a basis for the claims and as a representation basis for teaching one skilled in the art to variously employ the present invention in virtually any appropriately detailed structure.

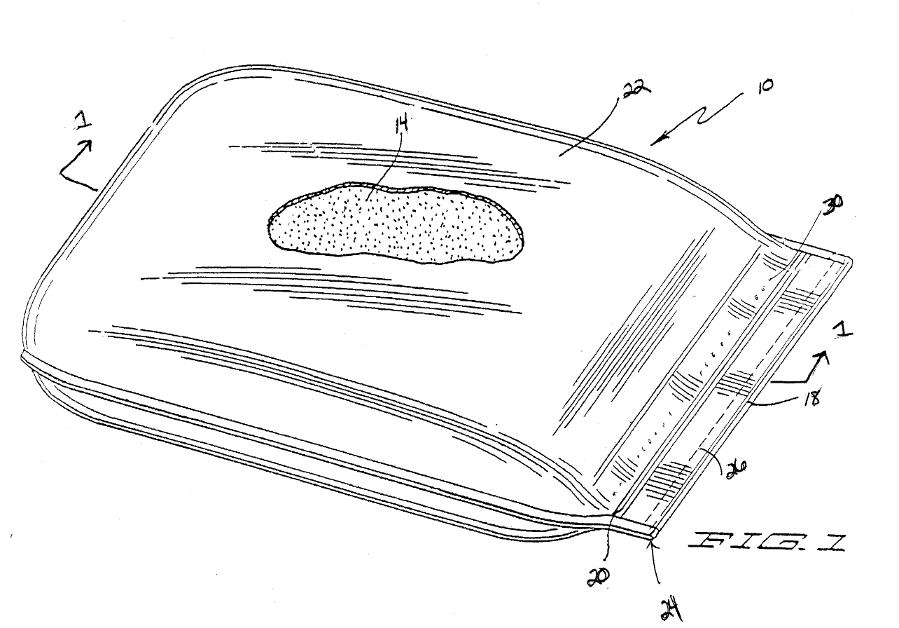

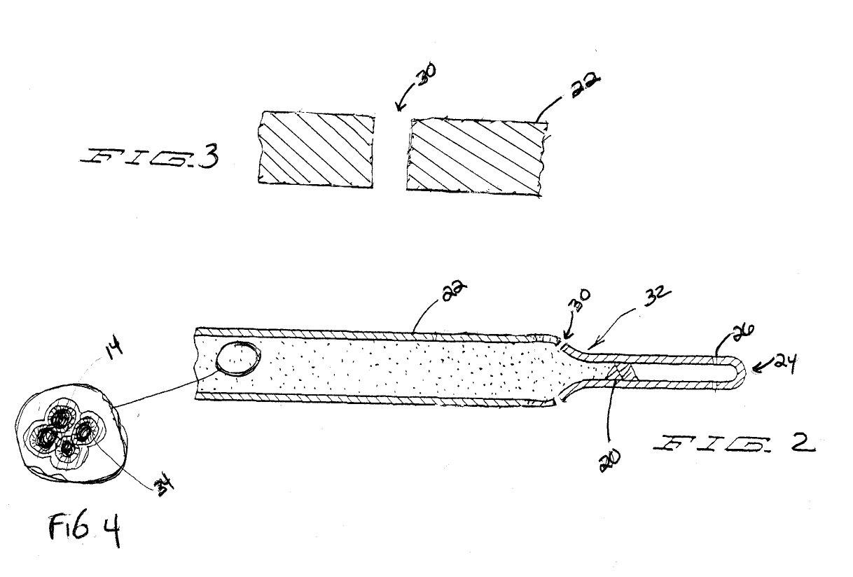

Referring now to FIGS. 1-4, wherein like elements are numbered consistently throughout, FIG. 1 illustrates one non-limiting embodiment of a storage system in accordance with the present invention which is suitable for effectively storing and sustaining microorganisms in a viable bio-active state prior to introduction into a closed aquatic environment, generally referenced here as 10.

As shown in FIG. 1, the storage system is shown lying on its side in a manner suitable for horizontal stacking. The storage system includes a breathable packaging material forming a package 12 constructed and arranged to envelop the substrate 14 in accordance with the present invention. The main body forms an interior region having a principal opening 18 which is sealed by any suitable sealing means 20.

The breathable packaging material is formed from durable material 22, such as polypropylene and/or polyethylene plastic. The material may be formed from a single polymer or a blend of various polymers so long as the package is sufficiently durable and resistant to tearing. The material may be formed from a single- or multi-layers. At least a portion of the breathable packaging material may be opaque, semi-opaque, transparent or translucent.

The breathable packaging material of the present invention may be flexible, semi-flexible, or rigid. According to a preferred, albeit non-limiting embodiment shown in FIG. 1, the breathable packaging material forms a flexible container which provides a lower profile suitable for stacking, which is often more desirable for shipping and retail sale. However, the package may be constructed and arranged to stand vertically as is known in the packaging art.

According to a preferred embodiment, an inner layer of the breathable packaging material is made from a hydrophobic (water repellant) material such as polytetrafluoroethylene which also helps prevent fluid permeation therethrough. Otherwise the interior portion of the breathable packaging material may be coated with a water repellant film, such as a fluorine-base water repellent coating or the like.

The sealing mechanism 20, shown in the closed position in FIG. 1, prevents the substrate from exiting the interior of the breathable packaging material. The sealing mechanism preferably comprises resealable sealing mechanism such as the zipper mechanism found on ZipLoc® storage bags. The sealing mechanism may also include an additional seal 24 formed during the manufacturing process by bonding the upper and lower package material across the opening 18. Such sealing techniques are well known in the art, these include heat, sonic welding, adhesive, pressure bonding, or the like. The package may also include an easy open feature such as score lines 26 through the material. The use of score lines are well known in the packaging art as they provide most consumers with the ability to open the package to access the contents therein without having to use scissors or the like.

According to a preferred, albeit non-limiting embodiment shown in the Figures, the package includes at least one perforation or opening 30 therein that allows air to permeate therethrough. The perforations are bored at a microscopic size using any suitable technique, such as laser machining. Examples of suitable lasers include, albeit are not limited to, a carbon dioxide, excimer, YAG, or the like. Examples of techniques for scoring packaging substrates using a laser are described in U.S. Pat. No. 5,630,308 to Guckenberger. The microperforations may be applied at any point during the manufacturing process of the present invention. That is, the perforations may be formed on the stock used to make the package or may be applied after the package has been formed and/or filled with substrate.

According to the embodiment shown in FIG. 1, the microperforations extend across the width of the package. The microsized perforations are formed on a portion of the package 32 near the opening. This allows the packages to be stacked one on top of the other without blocking the perforations. According to another embodiment not shown, the microperforations may be formed across the entire surface of the package. It is herein contemplated that microperforations could be formed in any variation, for example, the perforations may be formed randomly or uniformly distributed across a particular portion of the package. The shape of the perforation is not critical, they may be circular openings, slits, cuts, or the like. The microscopically sized perforations may be any size as long at they are sized in a manner effective to allow the passages of gases but not liquids or solids therethrough. For example, the dimension of the perforations may range from about 10 to about 150 microns across.

The breathable packaging material need only include sufficient porosity, due to either its inherent physical properties or owing to the presence of microperforations, necessary to allow gaseous exchange between the exterior and the interior of the package to prevent putrefaction and permit diffusion of gas through the interstitial fluid between the particles of substrate stored inside the package. For example, the breathable packaging material may contain about 300 to about 1500 microscopic perforations. It may desired to obscure the perforations by exterior graphics on the package so long as they are not blocked. By allowing the gas to continuously vent, the breathable packaging material of the present is invention capable of adjusting to pressure changes. Thus, the breathable packaging material of the present invention is able to be shipped at high altitudes without causing rupture or bursting.

The substrate refers to any suitable material to which the microorganisms can attach, grow, and form a biofilm upon. For example, as shown in FIG. 4, the substrate includes particles of sand, each of which are surrounded by the microorganism inside the biofilm 34. The substrate may be obtained directly from the marine and/or freshwater habitats, wherein the microorganisms already inhabit an established biofilm. However, the substrate need not come from aquatic environments. For example, the substrate may include spun fibrous material to which microorganisms may be added prior to insertion within the breathable packaging material or subsequent to being placed therein. These microorganisms will begin to colonize and form the biofilm on the substrate once inside the breathable packaging material. In either case, the time period between the manufacturing of the product and the purchase by the consumer is usually long enough (usually upwards of 6 months) so that the biofilm is adequately established by the microorganism prior to introduction into the aquarium to immediately begin biochemical cycling once added thereto.

In order for the microorganism to survive the storage period inside the breathable packaging material, the microorganisms must also remain hydrated. The substrate is introduced into the package with just enough liquid so as to form a thin film of liquid around the substrate and attached microorganisms, usually about 1 oz. per pound of substrate. This liquid may be separately introduced into the package and/or include liquid which remains attached thereto during the harvesting of the substrate from the marine or freshwater environment. The liquid inside the package remains essentially adhered onto the surface of the substrate particle by surface tension. The surface tension of all the substrate particles does not allow the liquid to pool or accumulate at the bottommost part of the package.

The accumulation of liquid at the bottom of the package is often viewed as objectionable to consumers, making the product undesirable for commercial use. Moreover, liquid accumulation is undesirable as it provides a larger amount of fluid that the air must diffuse through. The liquid accumulation at the bottom of the package is often viewed as undesirable to consumers making the product unattractive for commercial use. Thus, much of the microorganisms attached to the submersed substrate will putrefy as a result of oxygen starvation. The air which enters the package of the present invention is able to readily diffuse through the thin film of interstitial liquid and eventually to the attached microorganisms. This provides the aerobic conditions necessary for microbial respiration throughout the entire matrix of packaged substrate. Also, the air used by the microorganisms can escape from the interior of the package. Microbial respiration prevents putrefaction and spoilage inside the package.

In summary, the novel storage system of the present invention provides a commercial product which is encased within a breathable packaging material, utilizes a minimum amount of water and headspace air, and retains considerably more end product with minimal putrefaction therein. Since the package of the instant invention is not limited to the aforementioned parameters required by the prior art, the product is more easily manufactured, more compact (less storage space needed), and the cost of shipping is reduced.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and drawings/figures.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A storage system for containing a substrate bound population of microorganisms, and maintaining biological viability and hydration thereof, comprising in combination:
a substrate constructed and arranged to bind a plurality of hydrated and viable microorganisms thereon; and
a breathable packaging material which forms a containment device effective to envelope and contain a volume of said microorganism bound substrate, said containment device defining an interior space, and constructed and arranged to enable gaseous exchange therethrough of ambient atmospheric gases, in a manner effective to maintain a gaseous environment and a degree of hydration necessary to ensure said microorganisms remain viable during storage;
wherein at least a portion of said packaging material provides gaseous exchange between said ambient atmospheric gases and said interior space, so as to maintain aerobic conditions throughout said volume of substrate, and thereby prevent death and putrefaction of said bound microorganisms.

2. The storage system as set forth in claim 1, wherein said portion of said package material which provides gaseous exchange includes a plurality of perforations sized to permit said gaseous exchange while precluding permeation of moisture therethrough.

3. The storage system as set forth in claim 2, wherein said perforations are created by laser irradiation.

4. The storage system as set forth in claim 1, wherein said microorganisms include naturally occurring microorganisms.

5. The storage system as set forth in claim 1, wherein said microorganisms include genetically engineered microorganisms.

6. The storage system as set forth in claim 1, wherein said microorganisms reside within a biofilm.

7. The storage system as set forth in claim 1, wherein said substrate includes sand.

8. The storage system as set forth in claim 1, wherein said substrate includes spun-fibrous material.

9. The storage system as set forth in claim 1 wherein said microorganisms include aquatic microorganisms.

10. The storage system as set forth in claim 4 wherein said microorganisms include aquatic microorganisms.

11. The storage system as set forth in claim 5 wherein said microorganisms include aquatic microorganisms.

12. The storage system as set forth in claim 6 wherein said microorganisms include aquatic microorganisms.
